(19) United States
(12) Patent Application Publication
(10) Pub. No.: US 2024/0218291 A1
(43) Pub. Date: Jul. 4, 2024

(54) CONTACT LENS TREATING SOLUTION

(71) Applicant: Bausch + Lomb Ireland Limited, Dublin (IE)

(72) Inventors: Krista Fridman, Penfield, NY (US); Vicki Barniak, Fairport, NY (US); John Michael Duex, Livonia, NY (US); Elora Supersad, Rochester, NY (US); Olivia Culbertson, Hilton, NY (US)

(21) Appl. No.: 18/374,208

(22) Filed: Sep. 28, 2023

Related U.S. Application Data
(60) Provisional application No. 63/434,601, filed on Dec. 22, 2022.

(51) Int. Cl. C11D 3/00; C11D 3/04; C11D 3/22; C11D 3/30

(52) U.S. Cl. CPC C11D 3/0078; C11D 3/046; C11D 3/222; C11D 3/30

(57) ABSTRACT

A contact lens treating solution includes (a) one or more potassium salts selected from the group consisting of potassium chloride, potassium citrate, potassium hydroxide, potassium borate, potassium ethylenediaminetetraacetic acid and potassium phosphate, (b) an antimicrobial agent comprising alexidine or a salt or a free base thereof, and (c) optionally, one or more sodium salts selected from the group consisting of sodium chloride, sodium citrate, sodium hydroxide, sodium phosphate, sodium ethylenediaminetetraacetic acid and sodium borate, wherein the one or more potassium salts are present in an amount greater than an amount of the one or more sodium salts when present.

### PRIORITY

The present application claims priority to U.S. Provisional Patent Application Ser. No. 63/434,601, entitled “Contact Lens Treating Solution,” filed Dec. 22, 2022, the content of which is incorporated by reference herein in its entirety.

### BACKGROUND

Contact lenses are used by an increasing number of people as means of correcting vision and/or compensating for eye abnormalities. However, contact lenses must usually be inserted and removed daily with cleaning and disinfection between each wearing that requires sterile solutions and containers.

During wear and normal handling of contact lenses, microorganisms as well as biomolecules such as lipids, proteins, etc., can adhere to the contact lenses and contaminate the solutions and/or storage containers. Furthermore, a tear film that contains proteins, lipids, and even microorganisms can cover the surface of the eye. Any of these components found in the tear film, on the external surface of the eye or the surrounding skin, can be carried into the solutions and/or storage containers for the contact lens. Then, the microorganisms that multiplied in the solutions and/or storage containers can transfer to the eyes via the contact lens and become the pathogen that may cause eye infection resulting in impaired vision and blindness. Various solutions have been developed to clean these deposits and disinfect the microorganisms.

### SUMMARY

In accordance with an illustrative embodiment, a contact lens treating solution is provided comprising:
(a) one or more potassium salts selected from the group consisting of potassium chloride, potassium citrate, potassium hydroxide, potassium borate, potassium ethylenediaminetetraacetic acid and potassium phosphate,
(b) an antimicrobial agent comprising alexidine or a salt or a free base thereof, and
(c) optionally, one or more sodium salts selected from the group consisting of sodium chloride, sodium citrate, sodium hydroxide, sodium phosphate, sodium ethylenediaminetetraacetic acid and sodium borate, wherein the one or more potassium salts are present in an amount greater than an amount of the one or more sodium salts when present.

In accordance with another illustrative embodiment, a method of cleaning and disinfecting a contact lens is provided, the method comprising soaking the contact lens in a contact lens treating solution for a time period sufficient to clean and disinfect the contact lens, the contact lens treating solution comprising:
(a) one or more potassium salts selected from the group consisting of potassium chloride, potassium citrate, potassium hydroxide, potassium borate, potassium ethylenediaminetetraacetic acid and potassium phosphate,
(b) an antimicrobial agent comprising alexidine or a salt or a free base thereof, and
(c) optionally, one or more sodium salts selected from the group consisting of sodium chloride, sodium citrate, sodium hydroxide, sodium phosphate, sodium ethylenediaminetetraacetic acid and sodium borate, wherein the one or more potassium salts are present in an amount greater than an amount of the one or more sodium salts when present.

In accordance with yet another illustrative embodiment, a method for inhibiting adhesion of bacteria to a surface of a contact lens is provided, the method comprising contacting the surface of the contact lens with a contact lens treating solution comprising:
(a) one or more potassium salts selected from the group consisting of potassium chloride, potassium citrate, potassium hydroxide, potassium borate, potassium ethylenediaminetetraacetic acid and potassium phosphate,
(b) an antimicrobial agent comprising alexidine or a salt or a free base thereof, and
(c) optionally, one or more sodium salts selected from the group consisting of sodium chloride, sodium citrate, sodium hydroxide, sodium phosphate, sodium ethylenediaminetetraacetic acid and sodium borate, wherein the one or more potassium salts are present in an amount greater than an amount of the one or more sodium salts when present.

### DETAILED DESCRIPTION

The illustrative embodiments described herein are directed to contact lens treating solutions for cleaning, rinsing, storing and disinfecting a contact lens. For example, a “daily cleaner” containing various kinds of surfactants and disinfectants is recommended for daily use to remove most deposits and debris on contact lenses. In an approach to prevent protein deposits, contact lens treating solutions containing chemical agents such as cationic polymers were developed to prevent proteins from adhering to the contact lens surface of rigid gas permeable (RGP) and soft contacts lenses. In addition, solutions that wet the lenses before insertion in the eye are often required for both the hard and soft types of contact lenses, although their formulations have tended to be different based on their different properties. After the contact lens is inserted in the eye, ophthalmic solutions for rewetting, lubricating, and/or enhancing the comfort of the contact lens wearer can be applied to the eye by means of a drop dispenser.

Multipurpose solutions are popular because of the convenience of a single solution for cleaning, disinfecting and conditioning contact lenses immediately prior to insertion of the lens in the eye. Multipurpose solutions are also designed for use as a wetting agent, without rinsing, meaning that the solution must be ophthalmically safe for eye contact. This limits, to some extent, the type and concentration of both cleaning agents and biocides that can be employed in the solution as a preservative or disinfectant tends to be irritating to the eye. Additionally, the surface-active agents must not inhibit the wetting or conditioning function of the solution. Various solutions have been developed to clean these deposits and disinfect the microorganisms. However, there remains a need for improved solutions to clean these deposits and disinfect the microorganisms.

Illustrative embodiments described herein are directed to an improved contact lens treating solution for cleaning, rinsing, storing and disinfecting a contact lens. Various features of the compositions are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

The contact lens treating solution according to non-limiting illustrative embodiments disclosed herein includes at least (a) one or more potassium salts selected from the group consisting of potassium chloride, potassium citrate, potassium hydroxide, potassium borate, potassium ethylenediaminetetraacetic acid and potassium phosphate, (b) an antimicrobial agent comprising alexidine or a salt or a free base thereof, and (c) optionally, one or more sodium salts selected from the group consisting of sodium chloride, sodium citrate, sodium hydroxide, sodium phosphate, sodium ethylenediaminetetraacetic acid and sodium borate, wherein the one or more potassium salts are present in an amount greater than an amount of the one or more sodium salts when present.

Accordingly, a first component of the contact lens treating solution disclosed herein includes one or more potassium salts. In non-limiting illustrative embodiments, the one or more potassium salts include, for example, potassium chloride, potassium citrate, potassium hydroxide, potassium borate, potassium ethylenediaminetetraacetic acid such as the monopotassium, dipotassium, tripotassium, and tetrapotassium salts, potassium phosphate, etc.

In an illustrative embodiment, the one or more potassium salts are present in the contact lens treating solution in an amount ranging from about 0.02 to about 1.5 wt. %, based on the total weight of the contact lens treating solution. In another embodiment, the one or more potassium salts are present in the contact lens treating solution in an amount ranging from about 0.05 to about 0.9 wt. %, based on the total weight of the contact lens treating solution.

A second component of the contact lens treating solution disclosed herein includes alexidine or a salt or a free base thereof as an antimicrobial agent/disinfectant. As one skilled in the art will readily appreciate, alexidine is a non-polymer biguanide also known as 1, l′-hexamethylene-bis[5-(2-ethylhexyl)biguanide]. In an illustrative embodiment, the alexidine is present as alexidine per se, a salt of alexidine, e.g., alexidine HCl, alexidine free base, and mixtures thereof. The salts of alexidine can be either organic or inorganic and are typically disinfecting nitrates, acetates, phosphates, sulfates, halides and the like.

In an illustrative embodiment, alexidine is present in the contact lens treating solution in an amount ranging from about 0.0001 to about 0.0006 wt. %, based on the total weight of the contact lens treating solution. In another embodiment, alexidine is present in the contact lens treating solution in an amount ranging from about 0.0002 to about 0.0003 wt. %, based on the total weight of the contact lens treating solution.

In non-limiting illustrative embodiments, the contact lens treating solution disclosed herein can optionally contain one or more sodium salts selected from the group consisting of sodium chloride, sodium citrate, sodium hydroxide, sodium phosphate, sodium ethylenediaminetetraacetic acid and sodium borate. When one or more of these sodium salts are present in the contact lens treating solution, the one or more potassium salts discussed above will be present in an amount greater than an amount of the one or more sodium salts when present.

In an illustrative embodiment, the one or more sodium salts are present in the contact lens treating solution in an amount ranging from about 0.01 to about 1 wt. %, based on the total weight of the contact lens treating solution.

In one or more additional non-limiting illustrative embodiments, as may be combined with one or more of the preceding paragraphs, the contact lens treating solution disclosed herein can contain other non-polymeric biguanides in addition to alexidine. For example, in an illustrative embodiment, other non-polymeric biguanides in addition to alexidine include chlorhexidine, salts of chlorhexidine, and the like and mixtures thereof. The salts chlorhexidine can be either organic or inorganic and are typically disinfecting nitrates, acetates, phosphates, sulfates, halides and the like. The additional non-polymeric biguanides can be present in the contact lens treating solution in amounts similar to alexidine, e.g., an amount ranging from about 0.0001 to about 0.0006 wt. %, based on the total weight of the contact lens treating solution.

In one or more additional non-limiting illustrative embodiments, as may be combined with one or more of the preceding paragraphs, the contact lens treating solution can further contain one or more additional antimicrobial agents/disinfectants. In non-limiting illustrative embodiments, the one or more additional antimicrobial agents/disinfectants include one or more polyquaternium polymers or a salt thereof or free base thereof. Suitable one or more polyquaternium polymers for use herein include polyquaternium polymers having quaternary-amine-functional repeat units ranging from about 30 units to about 50,000 units. In one exemplary embodiment, the at least one polyquaternium polymer can have a quaternary-amine-functional repeat unit ranging from about 50 units to about 2,000 units. A “quaternary-amine-functional repeat unit” as used herein is understood to mean that the repeat unit comprises a quaternary-amine group in which a positively charged nitrogen atom is covalently bonded to four radicals (no hydrogen atoms) and ionically bonded to a negatively charged counterion such as a chloride.

The one or more polyquaternium polymers can have a weight average molecular weight Mw of from about 3,000 to about 5,000,000. In one exemplary embodiment, the at least one polyquaternium polymer can have a weight average molecular weight Mw of about 5,000 to about 500,000. In another exemplary embodiment, the one or more polyquaternium polymers can have a weight average molecular weight Mw of about 5,000 to about 200,000. In another exemplary embodiment, the one or more polyquaternium polymers can have a weight average molecular weight Mw of about 5,000 to about 50,000. In one exemplary embodiment, the one or more polyquaternium polymers can have a weight average molecular weight Mw of about 5,000 to about 30,000. In another exemplary embodiment, the one or more polyquaternium polymers can have a weight average molecular weight Mw of about 18,000 to about 24,000.

In non-limiting illustrative embodiments, the polyquaternium polymers useful herein may include copolymers in which the quaternary-amine-functional repeat units are derived from one or more of the following kinds of monomers: N,N-dimethyl-N-ethyl-aminoethyl acrylate and methacrylate, 2-methacryloxyethyltrimethylammonium, N-(3-methacrylamidopropyl)-N,N,N-trimethylammonium, 1-vinyl and 3-methyl-1-vinylimidazole, N-(3-acrylamido-3-methylbutyl)-N,N,N-trimethylammonium, N-(3-methacryloyloxy-2-hydroxypropyl)-N,N,N-trimethylammonium, their halides or other salt forms, and derivatives thereof, for example, involving the substitution, addition, or removal of alkyl groups such as alkyl groups having 1 to 6 carbon atoms. Quaternary-amine-functional repeat units can also be obtained as a reaction product or two or more compounds, for example, by the use of a strong alkylating agent such as 1,4-dichloro-2-butene which, for example, can be reacted with 1,4-bis[dimethylaminol]-2-butene and triethanolamine to produce a polymeric polyquaternary ammonium compound. Quaternary-amine-functional repeat units can also be made from other polymers, such as by the reaction of a trimethyl ammonium substituted epoxide with the hydroxy group of a hydroxyethylcellulose.

Suitable quaternary-amine-functional repeat units also include those found in polymeric ionenes and the like formed by a polycondensation reaction; in such repeat units, the nitrogens of the quaternary-amines are integral to the polymeric backbone and are situated between alkylene, oxyalkylene, or other segments.

In an exemplary embodiment, the nitrogens in the quaternary-amine-functional repeat units are part of a saturated or unsaturated heterocyclic ring, such as a five- or six-membered ring. In one embodiment, the polyquaternium polymer is a copolymer of a vinylimidazolium salt or a dimethyldiallyl ammonium salt. In one embodiment, up to about 90%, e.g., about 40% to about 90% by mole, of copolymerization-compatible comonomers not having a quaternary-amine-functionality may be copolymerized with the quaternary-amine-functional comonomers. Suitable comonomers include, for example, vinylpyrrolidone, acrylic acid, alkyl methacrylate, amides and amines such as acrylamide and N,N-dialkylaminoalkyl acrylate and methacrylate, hydroxyethylcellulose and copolymerization-compatible mixtures thereof. In one embodiment, an alkyl group has 1 to 6 carbon atoms.

Polyquaternium polymers as thus defined are a well-known class of polymers, many variations of which are commercially available. For example, a current CTFA International Cosmetic Ingredient Dictionary includes polyquaterniums designated as Polyquaternium-1 through Polyquaternium-68, a number of which, based on the present teachings, are useful in the illustrative embodiments disclosed herein. The polymerization techniques for the preparation of such materials are similarly well known to those skilled in the art and many variations of such techniques are similarly in practice in commerce. New variations of such polyquaternium polymers are in continuous commercial development, for example, various polymers having different combinations of the same or similar repeat units, different relative proportions of comonomers, and/or different molecular weights are in continuous commercial development.

In one embodiment, a polyquaternium polymer is Polyquaternium-1. Polyquaternium-1 is either commercially available from such sources as Stepan Inc. under the tradename Onamer® M or can be synthesized by well-known methods, see, for example, U.S. Pat. No. 4,027,020, the contents of which are incorporated by reference herein. If desired, the polymer can have alternative end groups such as hydroxyallylic end groups, aminoallylic end groups and diene end groups, see, for example, U.S. Pat. No. 7,705,112, the contents of which are incorporated by reference herein.

The one or more polyquaternium polymers suitably include an ophthalmologically suitable anionic organic or inorganic counterion. In an illustrative embodiment, a preferred counterion is chloride.

In certain embodiments, the cationic oligomer or polymer is characterized by a charge density that may be determined by methods known in the art, such as colloidal titration. In one embodiment, the charge density of the cationic oligomer or polymer is at least about 0.1 meq/g, in another embodiment at least about 2.5 meq/g, and in yet another embodiment, at least about 5 meq/g.

In an illustrative embodiment, the one or more polyquaternium polymers are present in the contact lens treating solution in an amount ranging from about 0.00005 to about 0.0003 wt. %, based on the total weight of the contact lens treating solution. In another embodiment, the one or more polyquaternium polymers are present in the contact lens treating solution in an amount ranging from about 0.0001 to about 0.0002 wt. %, based on the total weight of the contact lens treating solution.

Additional antimicrobial agents/disinfectants that can be used in the contact lens treating solution disclosed herein include, for example, a polymeric biguanide or a salt or free base thereof, a terpene or derivative thereof, a branched, glycerol monoalkyl ether, a branched, glycerol monoalkyl amine, a branched, glycerol monoalkyl sulphide, a fatty acid monoester, wherein the fatty acid monoester comprises an aliphatic fatty acid portion having six to fourteen carbon atoms, and an aliphatic hydroxyl portion, an amidoamine compound, and the like and combinations thereof.

Suitable polymeric biguanide antimicrobial agents include, for example, polymeric hexamethylene biguanides (PHMB) (commercially available from Zeneca, Wilmington, Del.), their polymers and water-soluble salts. In one embodiment, water-soluble polymeric biguanides for use herein can have a number average molecular weight of at least about 1,000 or a number average molecular weight from about 1,000 to about 50,000. Suitable water-soluble salts of the free bases include, for example, hydrochloride, borate, acetate, gluconate, sulfonate, tartrate and citrate salts. Generally, the hexamethylene biguanide polymers, also referred to as polyaminopropyl biguanide (PAPB), have number average molecular weights of up to about 100,000. Such compounds are known and are disclosed in U.S. Pat. No. 4,758,595, the contents of which are incorporated by reference herein.

PHMB is best described as a polymeric biguanide composition comprising at least three and preferably at least six biguanide polymers, which we refer to as PHMB-A, PHMB-CG and PHMB-CGA, the general chemical structures of which are depicted below.

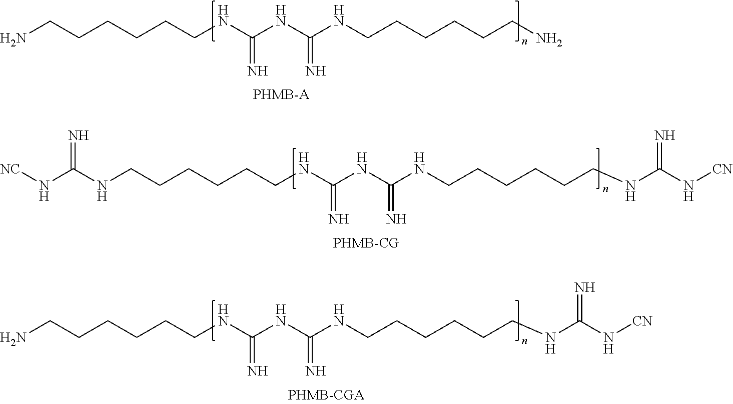

For each of these polymers, “n” represents the average number of repeating groups. A distribution of polymer length would exist for each of the polymers shown. The prior synthetic routes to PHMB provided a polymeric biguanide composition with about 50% by weight of the polymeric composition as PHMB-CGA, that is, having a cyanoguanidino end cap on one end and an amine on the other end, about 25% by weight PHMB-A and about 25% by weight PHMB-CG. Given this approximate weight ratio of the three major PHMB polymers above, the percentage of cyanoguanidino end caps is also about 50% of the total number of terminal groups. In this application we refer to this conventional polymeric biguanide composition as poly(hexamethylene biguanide) or PHMB.

A polymeric biguanide composition comprising less than about 18 mole % of terminal amine groups as measured by ¹³CNMR can also be used. The polymeric biguanide composition can also be characterized by a relative increase in the molar concentration of terminal guanidine groups or terminal cyanoguanidino groups. For example, in one embodiment, the biguanide composition comprises less than about 18 mole % of terminal amine groups and about 40 mol % or greater of terminal guanidine groups. In another embodiment, the biguanide composition comprises less than about 18 mole % of terminal amine groups and about 55 mol % or greater of terminal guanidine groups.

This biguanide composition is referred to as PHMB-CG*. Polymeric biguanide compositions are also referred to in the generic sense as “hexamethylene biguanides”, which one of ordinary skill in the art would recognize to include both PHMB as well as PHMB-CG*.

Suitable terpene antimicrobial agents include, for example, any monoterpene, sesquiterpene and/or diterpene or derivatives thereof. Acyclic, monocyclic and/or bicyclic mono-, sesqui- and/or diterpenes, and those with higher numbers of rings, can be used. A “derivative” of a terpene as used herein shall be understood to mean a terpene hydrocarbon having one or more functional groups such as terpene alcohols, terpene ethers, terpene esters, terpene aldehydes, terpene ketones and the like and combinations thereof. Here, both the trans and also the cis isomers are suitable. In one embodiment, the terpenes as well as the terpene moiety in the derivative can contain from 6 to about 100 carbon atoms or from about 10 to about 25 carbon atoms.

Representative examples of suitable terpene alcohol antimicrobial agents include verbenol, transpinocarveol, cis-2-pinanol, nopol, isoborneol, carbeol, piperitol, thymol, α-terpineol, terpinen-4-ol, menthol, 1,8-terpin, dihydro-terpineol, nerol, geraniol, linalool, citronellol, hydroxycitronellol, 3,7-dimethyl octanol, dihydro-myrcenol, tetrahydro-alloocimenol, perillalcohol, falcarindiol and the like and mixtures thereof.

Representative examples of suitable terpene ether and terpene ester antimicrobial agents include 1,8-cineole, 1,4-cineole, isobornyl methylether, rose pyran, α-terpinyl methyl ether, menthofuran, trans-anethole, methyl chavicol, allocimene diepoxide, limonene mono-epoxide, isobornyl acetate, nonyl acetate, α-terpinyl acetate, linalyl acetate, geranyl acetate, citronellyl acetate, dihydro-terpinyl acetate, meryl acetate and the like and mixtures thereof.

Representative examples of terpene aldehyde and terpene ketone antimicrobial agents include myrtenal, campholenic aldehyde, perillaldehyde, citronellal, citral, hydroxy citronellal, camphor, verbenone, carvenone, dihydro-carvone, carvone, piperitone, menthone, geranyl acetone, pseudo-ionone, α-ionine, iso-pseudo-methyl ionone, n-pseudo-methyl ionone, iso-methyl ionone, n-methyl ionone and the like and mixtures thereof. Any other terpene hydrocarbons having functional groups known in the art may be used herein in the inventive composition.

In an illustrative embodiment, suitable terpenes or derivatives thereof as antimicrobial agents include, but are not limited to, tricyclene, α-pinene, terpinolene, carveol, amyl alcohol, nerol, ß-santalol, citral, pinene, nerol, b-ionone, caryophillen (from cloves), guaiol, anisaldehyde, cedrol, linalool, d-limonene (orange oil, lemon oil), longifolene, anisyl alcohol, patchouli alcohol, α-cadinene, 1,8-cineole, ρ-cymene, 3-carene, ρ-8-mentane, trans-menthone, borneol, α-fenchol, isoamyl acetate, terpin, cinnamic aldehyde, ionone, geraniol (from roses and other flowers), myrcene (from bayberry wax, oil of bay and verbena), nerol, citronellol, carvacrol, eugenol, carvone, α-terpineol, anethole, camphor, menthol, limonene, nerolidol, farnesol, phytol, carotene (vitamin A₁), squalene, thymol, tocotrienol, perillyl alcohol, borneol, simene, carene, terpenene, linalool, 1-terpene-4-ol, zingiberene (from ginger) and the like and mixtures thereof.

In an illustrative embodiment, a suitable branched, glycerol monoalkyl ether antimicrobial agent is 3-[(2-ethylhexyl)oxy]-1,2-propanediol (EHOPD). In another embodiment, a suitable branched, glycerol monoalkyl amine antimicrobial agent is 3-[(2-ethylhexyl)amino]-1,2-propanediol (EHAPD). In another embodiment, a suitable branched, glycerol monoalkyl sulphide antimicrobial agent is 3-[(2-ethylhexyl)thio]-1,2-propanediol (EHSPD). In still another embodiment, the ophthalmic composition comprises any one mixture of EHOPD, EHAPD and EHSPD antimicrobial agents. The chemical structures of EHOPD, EHAPD and EHSPD are provided below.

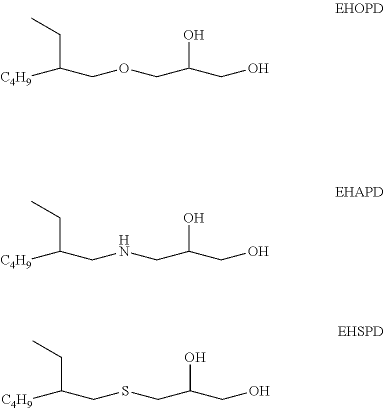

EHOPD is also referred to as octoxyglycerin and is sold under the tradename Sensiva® SC50 (Schülke & Mayr). EHOPD is a branched, glycerol monoalkyl ether known to be gentle to the skin, and to exhibit antimicrobial activity against a variety of Gram-positive bacteria such as Micrococcus luteus, Corynebacterium aquaticum, Corynebacterium flavescens, Corynebacterium callunae, and Corynebacterium nephredi. Accordingly, EHOPD is used in various skin deodorant preparations at concentrations between about 0.2 and 3 percent by weight. EHAPD can be prepared from 2-ethylhexylamine and 2,3-epoxy-1-propanediol using chemistry well known to those of ordinary skill in the art. EHSPD can be prepared from 2-ethylhexylthiol and 2,3-epoxy-1-propanediol using chemistry well known to those of ordinary skill in the art.

Suitable fatty acid monoester antimicrobial agents include, for example, fatty acid monoesters comprising an aliphatic fatty acid portion having six to fourteen carbon atoms, and an aliphatic hydroxyl portion. The term “aliphatic” refers to a straight or branched, saturated or unsaturated hydrocarbon having six to fourteen carbon atoms. In one embodiment, the aliphatic fatty acid portion is a straight chain, saturated or unsaturated hydrocarbon with eight to ten carbons. In another embodiment, the aliphatic fatty acid portion is a branched chain, saturated or unsaturated hydrocarbon with eight to ten carbons.

The aliphatic hydroxyl portion of the fatty acid monoester can be any aliphatic compound with at least one hydroxyl group. In addition, the aliphatic hydroxyl portion can have from three to nine carbons. The aliphatic hydroxyl portion can include, but is not limited to, propylene glycol, glycerol, a polyalkylene glycol, e.g., polyethylene glycol or polypropylene glycol, a cyclic polyol, e.g., sorbitan, glucose, mannose, sucrose, fructose, fucose and inisitol and derivatives thereof, and a linear polyol, e.g., mannitol and sorbitol and derivatives thereof and the like and mixtures thereof.

Suitable amidoamine antimicrobial agents include, for example, those amidoamines of the general formula:

$${R}^{15} - {( {OCH}_{2} {CH}_{2} )}_{m} - X - {( {CH}_{2} )}_{n} - Y$$

wherein R¹⁵ is a is C₆-C₃₀ saturated or unsaturated hydrocarbon including by way of example, a straight or branched, substituted or unsubstituted alkyl, alkylaryl, or alkoxyaryl group; m is zero to 16; n is 2 to 16; X is —C(O)—NR¹⁶— or —R¹⁶N—C(O)—; Y is —N(R¹⁷)₂ wherein each of R¹⁶ and R¹⁷ independently are hydrogen, a C₁-C₈ saturated or unsaturated alkyl or hydroxyalkyl, or a pharmaceutically acceptable salt thereof.

As one skilled in the art will readily appreciate, some of the amidoamines utilized in the contact lens treating solution disclosed herein are commercially available. For example, myristamidopropyl dimethylamine is commercially available from Alcon Inc. (Fort Worth, Tx.) under the tradename AldoxR; lauramidopropyl dimethylamine is commercially available from Inolex Chemical Company (Philadelphia, Pa.) under the tradename LEXAMINER L-13; and stearamidopropyl dimethylamine is also commercially available from Inolex Chemical Company as LEXAMINER S-13. The above-described amidoamines can be synthesized in accordance with known techniques, including those described in U.S. Pat. No. 5,573,726, the contents of which are incorporated by reference herein.

In illustrative embodiments, the foregoing one or more antimicrobial agents can be used in an amount that will at least partially reduce the microorganism population in the contact lens treating solution employed. If desired, the one or more antimicrobial agents may be employed in a disinfecting amount, which may reduce the microbial bioburden by, for example, at least two log orders in four hours and or by one log order in one hour. In one non-limiting illustrative embodiment, a disinfecting amount is an amount which will eliminate the microbial burden on a contact lens when used in regimen for the recommended soaking time (FDA Chemical Disinfection Efficacy Test-July, 1985 Contact Lens Solution Draft Guidelines).

In non-limiting illustrative embodiments, the foregoing antimicrobial agents/disinfectants can be present in the contact lens treating solution in an amount ranging from about 0.00005 to about 0.15 wt. %, based on the total weight of the contact lens treating solution. In another illustrative embodiment, the foregoing antimicrobial agents/disinfectants can be present in the contact lens treating solution in an amount ranging from about 0.0001 to about 0.001 wt. %, based on the total weight of the contact lens treating solution.

In one or more additional non-limiting illustrative embodiments, as may be combined with one or more of the preceding paragraphs, the contact lens treating solution disclosed herein can further include one or more surfactants. Suitable surfactants for use in the contact lens treating solution include one or more poloxamers, poloxamines and mixtures thereof. A representative example of a suitable poloxamer is a poloxamer block copolymer. One specific class of poloxamer block copolymers are those available under the trademark Pluronic (BASF Wyandotte Corp., Wyandotte, Mich.). Poloxamers include Pluronics and reverse Pluronics. Pluronics are a series of ABA block copolymers composed of poly(ethylene oxide)-poly(propylene oxide)-poly(ethylene oxide) blocks as generally represented in Formula I:

$${HO ( {C}_{2} {H}_{4} O )}_{a} {( {C}_{3} {H}_{6} O )}_{b} {( {C}_{2} {H}_{4} O )}_{a} H \qquad ( I )$$

wherein a is independently at least 1 and b is at least 1.

Reverse Pluronics are a series of BAB block copolymers, respectively composed of poly(propylene oxide)-poly(ethylene oxide)-poly(propylene oxide) blocks as generally represented in Formula II:

$${HO ( {C}_{3} {H}_{6} O )}_{b} {( {C}_{2} {H}_{4} O )}_{a} {( {C}_{3} {H}_{6} O )}_{b} H \qquad ( II )$$

wherein a is at least 1 and b is independently at least 1. The poly(ethylene oxide), PEO, blocks are hydrophilic, whereas the poly(propylene oxide), PPO, blocks are hydrophobic in nature. The poloxamers in each series have varying ratios of PEO and PPO which ultimately determines the hydrophilic-lipophilic balance (HLB) of the material, i.e., the varying HLB values are based upon the varying values of a and b, a representing the number of hydrophilic poly(ethylene oxide) units (PEO) being present in the molecule and b representing the number of hydrophobic poly(propylene oxide) units (PPO) being present in the molecule. In one embodiment, the poloxamer will have an HLB ranging from about 5 to about 24. In another embodiment, the poloxamer will have an HLB ranging from about 1 to about 5.

Poloxamers and reverse poloxamers have terminal hydroxyl groups that can be terminal functionalized. An example of a terminal functionalized poloxamer as discussed herein is poloxamer dimethacrylate (e.g., PluronicR F127 dimethacrylate) as disclosed in U.S. Patent Application Publication No. 2003/0044468 and U.S. Pat. No. 9,309,357, the contents of which are incorporated by reference herein. Other examples include glycidyl-terminated copolymers of polyethylene glycol and polypropylene glycol as disclosed in U.S. Pat. No. 6,517,933, the contents of which are incorporated by reference herein.

The poloxamer is functionalized to provide the desired reactivity at the end terminal of the molecule. The functionality can be varied and is determined based upon the intended use of the functionalized PEO- and PPO-containing block copolymers. That is, the PEO- and PPO-containing block copolymers are reacted to provide end terminal functionality that is complementary with the intended device forming monomeric mixture. The term block copolymer as used herein shall be understood to mean a poloxamer as having two or more blocks in their polymeric backbone(s).

In an illustrative embodiment, the one or more poloxamers are present in the contact lens treating solution in an amount ranging from about 0.001 to about 5.0 wt. %, based on the total weight of the contact lens treating solution. In another illustrative embodiment, the one or more poloxamers are present in the contact lens treating solution in an amount ranging from about 0.005 to about 1.0 wt. %, based on the total weight of the contact lens treating solution.

While the poloxamers and reverse poloxamers are considered to be difunctional molecules (based on the terminal hydroxyl groups), the poloxamines are in a tetrafunctional form, i.e., the molecules are tetrafunctional block copolymers terminating in primary hydroxyl groups and linked by a central diamine. One specific class of poloxamine block copolymers are those available under the trademark Tetronic (BASF). Poloxamines include Tetronic and reverse Tetronics. Poloxamines have the following general structure of Formula III:

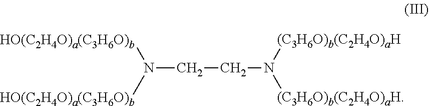

wherein a is independently at least 1 and b is independently at least 1.

The poloxamine can be functionalized to provide the desired reactivity at the end terminal of the molecule. The functionality can be varied and is determined based upon the intended use of the functionalized PEO- and PPO-containing block copolymers. That is, the PEO- and PPO-containing block copolymers are reacted to provide end terminal functionality that is complementary with the intended device forming monomeric mixture. The term block copolymer as used herein shall be understood to mean a poloxamine as having two or more blocks in their polymeric backbone(s).

In an illustrative embodiment, the one or more poloxamines are present in the contact lens treating solution in an amount ranging from about 0.001 to about 5.0 wt. %, based on the total weight of the contact lens treating solution. In another illustrative embodiment, the one or more poloxamines are present in the contact lens treating solution in an amount ranging from about 0.1 to about 1.2 wt. %, based on the total weight of the contact lens treating solution.

In one or more additional non-limiting illustrative embodiments, as may be combined with one or more of the preceding paragraphs, the contact lens treating solution disclosed herein can further contain one or more polysaccharides. In one embodiment, a polysaccharide comprises an anionic polysaccharide. Suitable anionic polysaccharides include, for example, hyaluronic acid or a salt thereof, e.g., sodium hyaluronate or potassium hyaluronate, chondroitin sulfate, chitosan, aloe vera, and carboxymethylcellulose. In one embodiment, a polysaccharide comprises a non-ionic polysaccharide. Suitable non-ionic polysaccharides include, for example, hemicellulose, hydroxypropyl methyl cellulose, methylcellulose, and ethylcellulose.

In an illustrative embodiment, the one or more polysaccharides are present in the contact lens treating solution in an amount ranging from about 0.01 to about 0.02 wt. %, based on the total weight of the contact lens treating solution.

In one or more additional non-limiting illustrative embodiments, as may be combined with one or more of the preceding paragraphs, the contact lens treating solution disclosed herein can further contain one or more comfort agents. Suitable comfort agents include, for example, polyols, antioxidants and complex carbohydrates. Suitable polyols include, for example, glucose, mannitol, erythritol, sorbitol, polyvinyl alcohol, maltose, glycerol, and trehalose. Suitable antioxidants include, for example, alpha-tocopherol and other water-soluble vitamin E moieties, ascorbic acid, ascorbyl glucoside, cysteine, carnosol, carnitine, epicatechin, gallic acid, resveratrol, ellagic acid, pycnogenol, lycopene, astaxanthin, coenzyme Q10, caffeic acid, hydroquinone monomethyl ether and butylated hydroxytoluene. Suitable complex carbohydrates include, for example, tremella polysaccharides and carboxymethyl cellulose.

In an illustrative embodiment, the one or more comfort agents are present in the contact lens treating solution in an amount ranging from about 0.1 to about 2.0 wt. %, based on the total weight of the contact lens treating solution. In another illustrative embodiment, the one or more comfort agents are present in the contact lens treating solution in an amount ranging from about 0.2 to about 1.5 wt. %, based on the total weight of the contact lens treating solution.

The contact lens treating solution disclosed herein may further contain one or more other components that are commonly present in contact lens treating solutions. In an illustrative embodiment, the contact lens treating solution can further include, for example, chelating agents; tonicity adjusting agents; pH adjusting agents, viscosity modifying agents, and demulcents and the like, which aid in making the contact lens treating solution more comfortable to the user and/or more effective for their intended use.

In an illustrative embodiment, suitable one or more chelating components can be employed to assist in the removal of lipid and protein deposits from the lens surface following daily use. Typically, the contact lens treating solution will include relatively low amounts, e.g., from about 0.005% to about 0.20% (w/v) of ethylenediaminetetraacetic acid (EDTA) or the corresponding metal salts thereof such as the disodium salt, Na₂EDTA.

In an illustrative embodiment, suitable tonicity adjusting agents include, for example, dextrose, calcium and magnesium chloride and the like and mixtures thereof. These tonicity adjusting agents are typically used individually in amounts ranging from about 0.01 to about 2.5% w/v. In an illustrative embodiment, the tonicity adjusting agents are used in amounts ranging from about 0.2 to about 1.5% w/v. The tonicity agents can be employed in an amount to provide a final effective osmotic value of at least about 150 mOsm/kg. In one embodiment, the tonicity adjusting agents are used in an amount to provide a final effective osmotic value of from about 150 to about 420 mOsm/kg. In an illustrative embodiment, the tonicity adjusting agents are used in an amount to provide a final effective osmotic value of from about 150 to about 350 mOsm/kg. In an illustrative embodiment, the tonicity adjusting agents are used in an amount to provide a final effective osmotic value of from about 160 to about 320 mOsm/kg.

In non-limiting illustrative embodiments, the contact lens treating solution disclosed herein can be formulated for direct instillation in the eye, including, by way of example, eye drop solutions and rewetting drops for rewetting a contact lens while worn as well as those that also qualify as a multi-purpose solution. In one non-limiting illustrative embodiment, the contact lens treating solution disclosed herein can be formulated as a composition instilled indirectly in the eye, such as contact lens treating solutions for treating the contact lens prior to the lens being inserted on the eye or a packaging solution for storing the lens.

The contact lens treating solution of the illustrative embodiments are physiologically compatible. Specifically, the contact lens treating solution should be “ophthalmically safe” for use with a contact lens, meaning that a contact lens treated with the contact lens treating solution is generally suitable and safe for direct placement on the eye without rinsing, that is, the contact lens treating solution is safe and comfortable for daily contact with the eye via a contact lens that has been wetted with the solution. An ophthalmically safe composition has a tonicity and pH that is compatible with the eye and comprises materials, and amounts thereof, that are non-cytotoxic according to ISO (International Standards Organization) standards and U.S. Food & Drug Administration (FDA) regulations. The compositions should be sterile in that the absence of microbial contaminants in the product prior to release must be statistically demonstrated to the degree necessary for such products.

In an illustrative embodiment, the pH of the contact lens treating solution disclosed herein may be a pH maintained within the range of about 4.0 to about 9.0, or about 5.0 to about 8.0, or about 6.0 to about 8.0, or about 6.5 to about 7.8. In one illustrative embodiment, a pH of the contact lens treating solution disclosed herein can be a pH greater than or equal to about 7.

In an illustrative embodiment, the osmolality of the contact lens treating solution disclosed herein may be an osmolality in the range of at least about 150 mOsm/kg, or at least about 200 mOsmol/kg, and up to about 420 mOsmol/kg. In an illustrative embodiment, the osmolality of the contact lens treating solution disclosed herein may be an osmolality of about 150 to about 420 mOsm/kg. In another illustrative embodiment, the osmolality of the contact lens treating solution disclosed herein may be an osmolality of about 150 to about 350 mOsm/kg. In another illustrative embodiment, the osmolality of the contact lens treating solution disclosed herein may be an osmolality of about 160 to about 320 mOsm/kg. In one illustrative embodiment, the osmolality of the contact lens treating solution disclosed herein may be an osmolality of about 300 to about 400 mOsm/kg. In another illustrative embodiment, the osmolality of the contact lens treating solution disclosed herein may be an osmolality of about 350 to about 400 mOsm/kg. The contact lens treating solution is substantially isotonic or hypertonic (for example, slightly hypertonic) and is ophthalmically acceptable.

The contact lens treating solution disclosed herein may be in the form of drops and are useful as a component of a contact lens cleaning, disinfecting or conditioning composition containing such materials. In non-limiting illustrative embodiments, the contact lens treating solution disclosed herein may be formulated as a “multi-purpose solution”. A multi-purpose solution is useful for cleaning, disinfecting, storing, and rinsing a lens, particularly soft contact lenses. Multi-purpose solutions do not exclude the possibility that some wearers, for example, wearers particularly sensitive to chemical disinfectants or other chemical agents, may prefer to rinse or wet a contact lens with another solution, for example, a sterile saline solution prior to insertion of the lens. The term “multi-purpose solution” also does not exclude the possibility of periodic cleaners not used on a daily basis or supplemental cleaners for further removing proteins, for example, enzyme cleaners, which are typically used on a weekly basis. By the term “cleaning” is meant that the solution contains one or more agents in sufficient concentrations to loosen and remove loosely held lens deposits and other contaminants on the surface of a contact lens, which may be used in conjunction with digital manipulation (e.g., manual rubbing of the lens with a solution) or with an accessory device that agitates the solution in contact with the lens, for example, a mechanical cleaning aid.

Traditionally, multi-purpose solutions on the market have required a regimen involving mechanical rubbing of the lens with the multi-purpose solution, in order to provide the required disinfection and cleaning. Such a regimen is required under governmental regulatory authorities (e.g., the FDA) for a Chemical Disinfection System that does not qualify as a Chemical Disinfecting Solution. In one embodiment, it is possible to formulate a cleaning and disinfecting solution that, on one hand, is able to provide improved cleaning and disinfection and, on the other hand, is gentle enough to be used as a wetting agent, e.g., as an eye drop. In one embodiment, a contact lens treating solution disclosed herein is formulated to meet the requirements of the FDA or ISO Stand-Alone Procedure for contact lens disinfecting products.

Accordingly, in a non-limiting illustrative embodiment, a method of cleaning and disinfecting a contact lens includes soaking the contact lens in a contact lens treating solution disclosed herein for a time period sufficient to clean and disinfect the contact lens. A suitable time period can include at least about 30 seconds, or from about 2 to about 12 hours, or from about 2 to about 4 hours.

Alternatively, a rub protocol would include each of the above steps plus the step of adding a few drops of the contact lens treating solution to each side of the lens, followed by gently rubbing the surface between one's fingers for about 3 to about 10 seconds. The lens can then be, optionally rinsed, and subsequently immersed in the contact lens treating solution for a suitable time period, e.g., a few minutes or several hours such as at least two hours. The lenses are removed from the lens storage case and repositioned on the eye.

The type of contact lens to be contacted with the contact lens treating solutions disclosed herein is not critical and any contact lens is contemplated. Representative examples of such lenses include, but are not limited to, soft contact lenses, e.g., a soft, hydrogel lens; soft, non-hydrogel lens and the like, hard contact lenses, e.g., a hard, gas permeable lens material and the like, rigid gas permeable (RGP) lenses, intraocular lenses, overlay lenses, and the like. As is understood by one skilled in the art, a lens is considered to be “soft” if it can be folded back upon itself without breaking. Any material known to produce a contact lens can be used herein. For example, the preservative-free contact lens treating solutions can be used with (1) hard lenses formed from materials prepared by polymerization of acrylic esters, such as poly(methyl methacrylate) (PMMA), (2) RGP lenses formed from silicone acrylates and fluorosilicone methacrylates, and (3) soft hydrogel contact lenses made of a hydrogel polymeric material, such as a silicone hydrogel, with a hydrogel being defined as a crosslinked polymeric system containing water in an equilibrium state.

In general, hydrogels exhibit excellent biocompatibility properties, i.e., the property of being biologically or biochemically compatible by not producing a toxic, injurious or immunological response in a living tissue. Representative conventional hydrogel contact lens materials are made by polymerizing a monomer mixture comprising at least one hydrophilic monomer, such as (meth)acrylic acid, 2-hydroxyethyl methacrylate (HEMA), glyceryl methacrylate, N,N-dimethacrylamide, and N-vinylpyrrolidone (NVP). In the case of silicone hydrogels, the monomer mixture from which the copolymer is prepared further includes a silicone-containing monomer, in addition to the hydrophilic monomer. Generally, the monomer mixture will also include a crosslinking monomer such as ethylene glycol dimethacrylate, tetraethylene glycol dimethacrylate, and methacryloxyethyl vinylcarbonate. Alternatively, either the silicone-containing monomer or the hydrophilic monomer may function as a crosslinking agent.

The following examples are provided to enable one skilled in the art to practice the invention and are merely illustrative. The examples should not be read as limiting the scope of the illustrative embodiments as defined in the claims. In the examples, the following abbreviations are used.
TRIS: tromethamine buffer.
Na₂EDTA: ethylenediaminetetraacetic acid disodium salt.
PAPB: polyaminopropyl biguanide.
Polyquaternium-1: a polyquaternium polymer represented by the following structure:

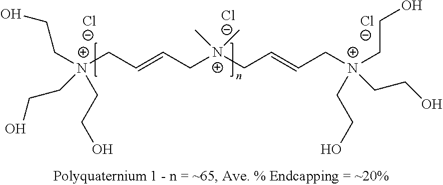

### Example 1 and Comparative Example A

A contact lens treating solution was made by mixing the following components, listed in Table 1 at amounts per weight.

TABLE 1

| Ingredient | Ex. 1 | Comp. Ex. A |
|---|---|---|
| Tris | 0.50% | 0.50% |
| Citric acid | 0.20% | 0.20% |
| Potassium Chloride | 0.17% | — |
| Sodium Chloride | — | 0.13% |
| Poloxamine 1107 | 1.00% | 1.00% |
| Poloxamer 181 | 0.01% | 0.01% |
| Glycerol | 0.50% | 0.50% |
| Erythritol | 1.00% | 1.00% |
| Na₂EDTA | 0.10% | 0.10% |
| Alexidine | 2.5 ppm | 2.5 ppm |
| Polyquaternium-1 | 1.5 ppm | 1.5 ppm |
| pH | 7.7 | 7.7 |
| Osmolality, mOsm/kg | 250 | 250 |

### Example 2 and Comparative Example B

A contact lens treating solution was made by mixing the following components, listed in Table 2 at amounts per weight.

TABLE 2

| Ingredient | Ex. 2 | Comp. Ex. B |
|---|---|---|
| Tris | 0.20% | 0.20% |
| Tris HCl | 0.45% | 0.45% |
| Glycylglycine | 0.30% | 0.30% |
| Potassium Chloride | 0.14% | — |
| Sodium Chloride | — | 0.11% |
| Poloxamine 1107 | 1.00% | 1.00% |
| Poloxamer 181 | 0.01% | 0.01% |
| Glycerol | 0.50% | 0.50% |
| Erythritol | 0.50% | 0.50% |
| Na₂EDTA | 0.10% | 0.10% |
| Alexidine | 2.5 ppm | 2.5 ppm |
| Polyquaternium-1 | 1.5 ppm | 1.5 ppm |
| pH | 7.7 | 7.7 |
| Osmolality, mOsm/kg | 240 | 240 |

### Testing

The contact lens treating solutions of Examples 1 and 2 and Comparative Examples A and B were tested for stand-alone biocidal efficacy. The “Stand-Alone Procedure for Disinfecting Products” is based on the Disinfection Efficacy Testing for Products dated May 1, 1997, prepared by the U.S. Food and Drug Administration, Division of Ophthalmic Devices and ISO 14729. The stand-alone test challenges a disinfecting product with a standard inoculum of a representative range of microorganisms and established the extent of viability loss at predetermined time intervals comparable with those during which the product may be used. The primary criteria for a given disinfection period (corresponding to a potential minimum recommended disinfection period) is that the number of bacteria recovered per milliliter (mL) must be reduced by a mean value of not less than 3.0 logs within the given disinfection period. The number of mold and yeast recovered per mL must be reduced by a mean value of not less than 1.0 log within the minimum recommended disinfection time with no increase at four times the minimum recommended disinfection time. The antimicrobial efficacy of each of the various compositions were evaluated in the presence of organic soil.

The stand-alone biocidal test was carried out as follows:

Microbial challenge inoculums were prepared using Staphylococcus aureus (ATCC 6538), Pseudomonas aeruginosa (ATCC 9027), Serratia marcescens (ATCC 13880), Candida albicans (ATCC 10231) and Fusarium solani (ATCC 36031). The challenge organisms were transferred onto a recommended agar and incubated for an appropriate duration and temperature. The cultures were harvested using sterile Dulbecco's Phosphate Buffered Saline plus 0.05 percent w/v polysorbate 80 (DPBST) or a suitable diluent and transferred to a suitable vessel. Spore suspensions were filtered through sterile glass wool to remove hyphal fragments. Serratia marcescens, as appropriate, was filtered through a 1.2 μm filter to clarify the suspension. After harvesting, the suspension was centrifuged at no more than 5000×g for a maximum of 30 minutes at a temperature of 20-25° C. The supernatant was decanted and resuspended in DPBST or other suitable diluent 1×10⁷ to 1×10⁸ cfu/ml.

The appropriate organism concentration was estimated by measuring the turbidity of the suspension, for example, using a spectrophotometer at a preselected wavelength, for example, 490 nm. One tube was prepared containing a minimum of 10 mL of test solution per challenge organism. An inoculum control (IC) was made by dispersing an identical aliquot of the inoculum into a suitable diluent (DPBST) using the same volume as in the test sample. The IC for each challenge organism was serially diluted and plated with the appropriate agar at the start of the test (T=0). Each tube of the solution to be tested was inoculated with a suspension of the test organism sufficient to provide a final count of 1×10⁵ to 1×10⁶ cfu/mL, the volume of the inoculum not exceeding 1 percent of the sample volume. Dispersion of the inoculum was ensured by adequately mixing the sample (e.g., by vortexing each tube a minimum of 5 seconds). The inoculated product was stored at 20-25° C. Aliquots in the amount of 1.0 mL are taken of the inoculated product for determination of viable counts after certain time period of disinfection.

The suspensions were mixed well by vortexing vigorously for at least 5 sec. The 1.0 mL aliquots removed at the specified time intervals were subjected to a suitable series of decimal dilutions in validated neutralizing media. The suspensions were mixed vigorously and incubated for a suitable period of time (minimum of 10 minutes and not exceeding 1 hour prior to plating) to allow for neutralization of the microbial agent. The viable count of organisms was determined in appropriate dilutions by preparation of duplicate plates of Trypticase Soy Agar (TSA) for bacteria and Sabouraud Dextrose Agar (SDA) for mold and yeast. The bacterial recovery plates were incubated at 30-35ºC for two to four days. The yeast recovery plates were incubated at 20-25° C. or 30-35° C. for three to five days. The mold recovery plates were incubated at 20-25° C. for four to seven days. The average number of colony forming units (cfu) was determined on countable plates. Countable plates refer to 30 to 300 cfu/plates for bacteria and yeast, and 8 to 80 cfu/plate for mold except when colonies were observed only for the 10° or 10-1 dilution plates. The microbial reduction was then calculated at the specified time points. In order to demonstrate the suitability of the medium used for growth of the test organisms and to provide an estimation of the initial inoculum concentration, inoculum controls were prepared by dispersing an identical aliquot of the inoculum into a suitable diluent used to suspend the organism listed above. Following inoculation in a validated neutralizing broth and incubation for an appropriate period of time, the inoculum control must be between 1.0×10⁵ and 1.0×10⁶ cfu/mL.

The log reduction values of the 4-hour disinfection efficacy test for the contact lens treating solutions of Examples 1 and 2 and Comparative Examples A and B are set forth below in Table 3.

TABLE 3

| Ex./Comp. Ex. | S.a. | P.a. | S.m. | C.a. | F.s. |
|---|---|---|---|---|---|
| Ex. 1 | >4.7 | >4.6 | >4.8 | 4.0 | >4.7 |
| Comp. Ex. A | >4.7 | >4.6 | >4.8 | 2.6 | >4.7 |
| Ex. 2 | >4.6 | >4.7 | >4.7 | 4.0 | >4.5 |
| Comp. Ex. B | >4.6 | >4.7 | >4.7 | 2.8 | >4.5 |

Each of the contact lens treating solutions of Examples 1 and 2 showed a significantly improved disinfection efficacy for the Candida albicans species as compared to the contact lens treating solutions of Comparative Examples A and B.

### Example 3 and Comparative Example C

A contact lens treating solution was made by mixing the following components, listed in Table 4 at amounts per weight.

TABLE 4

| Ingredient | Ex. 3 | Comp. Ex. C |
|---|---|---|
| Tris | 0.50% | 0.50% |
| Citric Acid | 0.20% | 0.20% |
| Potassium Chloride | 0.17% | — |
| Sodium Chloride | — | 0.13% |
| Poloxamine 1107 | 1.00% | 1.00% |
| Poloxamer 181 | 0.01% | 0.01% |
| Glycerol | 0.50% | 0.50% |
| Erythritol | 1.00% | 1.00% |
| Na₂EDTA | 0.10% | 0.10% |
| Alexidine | 2.5 ppm | 2.5 ppm |
| Polyquaternium-1 | 1.5 ppm | 1.5 ppm |
| pH | 7.7 | 7.7 |
| Osmolality, mOsm/kg | 245 | 245 |

### Example 4 and Comparative Example D

A contact lens treating solution was made by mixing the following components, listed in Table 5 at amounts per weight.

TABLE 5

| Ingredient | Ex. 4 | Comp. Ex. D |
|---|---|---|
| Tris | 0.50% | 0.50% |
| Citric Acid | 0.20% | 0.20% |
| Potassium Chloride | 0.17% | — |
| Sodium Chloride | — | 0.13% |
| Poloxamine 1107 | 1.00% | 1.00% |
| Poloxamer 181 | 0.01% | 0.01% |
| Glycerol | 0.50% | 0.50% |
| Erythritol | 1.00% | 1.00% |
| Na₂EDTA | 0.10% | 0.10% |
| Alexidine | 2.5 ppm | 2.5 ppm |
| pH | 7.7 | 7.7 |
| Osmolality, mOsm/kg | 245 | 245 |

### Comparative Examples E-H

A contact lens treating solution was made by mixing the following components, listed in Table 6 at amounts per weight.

TABLE 6

| | Comp. | Comp. | Comp. | Comp. |
|---|---|---|---|---|
| Ingredient | Ex. E | Ex. F | Ex. G | Ex. H |
| Tris | 0.50% | 0.50% | 0.50% | 0.50% |
| Citric Acid | 0.20% | 0.20% | 0.20% | 0.20% |
| Potassium Chloride | 0.17% | — | 0.17% | — |
| Sodium Chloride | — | 0.13% | — | 0.13% |
| Poloxamine 1107 | 1.00% | 1.00% | 1.00% | 1.00% |
| Poloxamer 181 | 0.01% | 0.01% | 0.01% | 0.01% |
| Glycerol | 0.50% | 0.50% | 0.50% | 0.50% |
| Erythritol | 1.00% | 1.00% | 1.00% | 1.00% |
| Na₂EDTA | 0.10% | 0.10% | 0.10% | 0.10% |
| Polyquaternium-1 | 1.5 ppm | 1.5 ppm | — | — |
| PAPB | — | — | 1.5 ppm | 1.5 ppm |
| pH | 7.7 | 7.7 | 7.7 | 7.7 |
| Osmolality, mOsm/kg | 245 | 245 | 245 | 245 |

### Testing

The contact lens treating solutions of Examples 3 and 4 and Comparative Examples C-H were tested using the disinfection efficacy test as described above. The log reduction values of the 4-hour disinfection efficacy test are shown below in Table 7.

TABLE 7

| Ex./Comp. Ex. | S.a. | P.a. | S.m. | C.a. | F.s. |
|---|---|---|---|---|---|
| Ex. 3 | >4.6 | >4.6 | >4.7 | 3.4 | >4.6 |
| Comp. Ex. C | >4.6 | >4.6 | >4.7 | 2.4 | >4.6 |
| Ex. 4 | >4.6 | >4.6 | 4.7 | 3.2 | >4.6 |
| Comp. Ex. D | 3.5 | >4.6 | >4.7 | 2.2 | >4.6 |
| Comp. Ex. E | >4.6 | >4.6 | 4.7 | 0.8 | >4.6 |
| Comp. Ex. F | 4.4 | >4.6 | >4.7 | 0.8 | 4.4 |
| Comp. Ex. G | >4.6 | >4.6 | 4.4 | 0.9 | >4.6 |
| Comp. Ex. H | >4.6 | >4.6 | 4.7 | 1.0 | 4.2 |

Each of the contact lens treating solutions of Examples 3 and 4 showed a significantly improved disinfection efficacy for the Candida albicans species as compared to the contact lens treating solutions of Comparative Examples C-H. When comparing the contact lens treating solutions of Examples 3 and 4 with respective contact lens treating solutions of Comparative Examples C and D, it can be seen that the inclusion of potassium chloride in a contact lens treating solution containing alexidine for Examples 3 and 4 showed a significantly improved disinfection efficacy for the Candida albicans species as compared to the inclusion of sodium chloride in a solution containing alexidine for Comparative Examples C and D. Comparative Examples E and F show that the inclusion of potassium chloride in a contact lens treating solution containing Polyquaternium-1 alone without alexidine does not improve disinfection efficacy for the Candida albicans species. Comparative Examples G and H also show that the inclusion of potassium chloride in a contact lens treating solution containing PAPB alone without alexidine as compared to the inclusion of sodium chloride in a contact lens treating solution containing PAPB alone without alexidine also does not affect the disinfection efficacy.

### Example 5 and Comparative Example I

A contact lens treating solution was made by mixing the following components, listed in Table 8 at amounts per weight.

TABLE 8

| Ingredient | Ex. 5 | Comp. Ex. I |
|---|---|---|
| Tris | 0.13% | 0.13% |
| Tris HCl | 0.45% | 0.45% |
| Glycylglycine | 0.30% | 0.30% |
| Potassium Chloride | 0.21% | — |
| Sodium Chloride | — | 0.16% |
| Poloxamine 1107 | 1.00% | 1.00% |
| Poloxamer 181 | 0.01% | 0.01% |
| Glycerol | 0.50% | 0.50% |
| Erythritol | 0.40% | 0.40% |
| Na₂EDTA | 0.10% | 0.10% |
| Sodium Hyaluronate | 0.01% | 0.01% |
| Alexidine | 2.5 ppm | 2.5 ppm |
| Polyquaternium-1 | 1.5 ppm | 1.5 ppm |
| pH | 7.5 | 7.5 |
| Osmolality, mOsm/kg | 245 | 245 |

The contact lens treating solutions of Example 5 and Comparative Example I were tested using the disinfection efficacy test described above. The log reduction values of the 4-hour disinfection efficacy test are shown below in Table 9.

### Testing

TABLE 9

| Ex./Comp. Ex. | S.a. | P.a. | S.m. | C.a. | F.s. |
|---|---|---|---|---|---|
| Ex. 5 | 4.1 | >4.6 | 4.5 | 3.4 | >4.6 |
| Comp. Ex. I | >4.6 | >4.6 | 4.7 | 2.6 | >4.6 |

The contact lens treating solution of Example 5 showed a significantly improved disinfection efficacy for the Candida albicans species as compared to the contact lens treating solution of Comparative Example I. Thus, it can be seen that the inclusion of potassium chloride in a contact lens treating solution containing alexidine for Example 5 performed significantly better as compared to the inclusion of sodium chloride in a contact lens treating solution containing alexidine for Comparative Example I.

### Example 6 and Comparative Example J-L

A contact lens treating solution was made by mixing the following components, listed in Table 10 at amounts per weight.

TABLE 10

| | | Comp. | Comp. | Comp. |
|---|---|---|---|---|
| Ingredient | Ex. 6 | Ex. J | Ex. K | Ex. L |
| Boric acid | 0.50% | 0.50% | 0.50% | 0.50% |
| Sodium borate | 0.08% | 0.08% | 0.08% | 0.08% |
| Na₂EDTA | 0.10% | 0.10% | 0.10% | 0.10% |
| Potassium Chloride | 0.65% | — | 0.65% | — |
| Sodium Chloride | — | 0.51% | — | 0.51% |
| Poloxamine 1107 | 0.80% | 0.80% | 0.80% | 0.80% |
| Alexidine | 2.5 ppm | 2.5 ppm | — | — |
| Polyquaternium-1 | 1.5 ppm | 1.5 ppm | — | — |
| PAPB | — | — | 1.4 ppm | 1.4 ppm |
| pH | 7.3 | 7.3 | 7.3 | 7.3 |
| Osmolality, mOsm/kg | 260 | 260 | 260 | 260 |

The contact lens treating solutions of Example 6 and Comparative Examples J-L were tested using the disinfection efficacy test described above. The log reduction values of the 4-hour disinfection efficacy test are shown below in Table 11.

### Testing

TABLE 11

| Ex./Comp. Ex. | S.a. | P.a. | S.m. | C.a. | F.s. |
|---|---|---|---|---|---|
| Ex. 6 | >4.6 | >4.6 | >4.7 | 2.6 | 3.4 |
| Comp. Ex. J | >4.6 | >4.6 | >4.7 | 1.2 | 2.4 |
| Comp. Ex. K | >4.6 | >4.6 | 4.2 | 2.3 | 4.4 |
| Comp. Ex. L | >4.6 | >4.6 | >4.7 | 2.1 | 3.9 |

When comparing the contact lens treating solutions of Example 6 with the contact lens treating solution of Comparative Example J, it can be seen that the inclusion of potassium chloride in a contact lens treating solution containing alexidine performed significantly better as compared to the inclusion of sodium chloride. Comparative Examples K and L show that the inclusion of potassium chloride in a contact lens treating solution containing PAPB alone without alexidine as compared to the inclusion of sodium chloride in a contact lens treating solution containing PAPB alone without alexidine also does not affect the disinfection efficacy.

### Examples 7-9 and Comparative Examples M-O

A contact lens treating solution was made by mixing the following components, listed in Table 12 at amounts per weight.

TABLE 12

| | | | | Comp. | Comp. | Comp. |
|---|---|---|---|---|---|---|
| Ingredient | Ex. 7 | Ex. 8 | Ex.9 | Ex. M | Ex. N | Ex. O |
| Tris | 0.24% | — | — | 0.24% | | — |
| Tris HCl | | 0.08% | | | 0.08% | |
| Sodium Citrate | — | — | — | 0.45% | — | — |
| Sodium Phosphate | | — | — | | — | 0.03% |
| Monobasic | | | | | | |
| Sodium Phosphate | — | — | — | — | 0.60% | 0.60% |
| Dibasic | | | | | | |
| Potassium Citrate | 0.49% | — | — | — | — | — |
| Potassium Phosphate | | — | 0.03% | | — | |
| Monobasic | | | | | | |
| Potassium Phosphate | — | 0.51% | 0.51% | — | — | — |
| Dibasic | | | | | | |
| Potassium Chloride | 0.23% | 0.28% | 0.28% | 0.23% | 0.28% | 0.28% |
| Na₂EDTA | 0.10% | 0.10% | 0.10% | 0.10% | 0.10% | 0.10% |
| Poloxamine 1107 | 1.00% | 1.00% | 1.00% | 1.00% | 1.00% | 1.00% |
| Glycerol | 0.94% | 0.80% | 0.50% | 0.94% | 0.80% | 0.50% |
| Erythritol | — | — | 0.50% | — | — | 0.50% |
| Alexidine | 1.5 ppm | 1.5 ppm | 1.5 ppm | 1.5 ppm | 1.5 ppm | 1.5 ppm |
| pH | 7.5 | 7.7 | 7.5 | 7.5 | 7.7 | 7.5 |
| Osmolality, mOsm/kg | 240 | 240 | 245 | 240 | 245 | 250 |

### Testing

The contact lens treating solutions of Examples 7-9 and Comparative Examples M-O were tested using the disinfection efficacy test described above. The log reduction values of the 4-hour disinfection efficacy test are shown below in Table 13.

TABLE 13

| Ex./Comp. Ex. | S.a. | S.m. | C.a |
|---|---|---|---|
| Ex. 7 | 3.1 | >4.6 | 1.8 |
| Ex. 8 | 4.2 | >4.6 | 1.9 |
| Ex. 9 | 3.2 | >4.6 | 1.9 |
| Comp. Ex. M | 2.8 | >4.6 | 1.2 |
| Comp. Ex. N | 3.0 | >4.6 | 0.9 |
| Comp. Ex. O | 2.9 | >4.6 | 0.9 |

The contact lens treating solutions of Examples 7-9 containing respective potassium salt buffering agents with alexidine demonstrating improved disinfection efficacy against Staphylococcus aureus and Candida albicans as compared to the contact lens treating solutions of Comparative Examples M-O containing the equivalent sodium salt buffering agents with alexidine.

### Example 11 and Comparative Example S

A contact lens treating solution was made by mixing the following components, listed in Table 14 at amounts per weight.

TABLE 14

| Ingredient | Ex. 10 | Comp. Ex. P |
|---|---|---|
| Potassium Citrate | 0.66% | — |
| Sodium Citrate | — | 0.60% |
| Potassium Chloride | 0.10% | — |
| Sodium Chloride | — | 0.08% |
| Boric acid | 0.90% | 0.90% |
| Sodium borate | 0.17% | 0.17% |
| Na₂EDTA | 0.10% | 0.10% |
| Poloxamine 1107 | 1.00% | 1.00% |
| Alexidine | 2.0 ppm | 2.0 ppm |
| PAPB | 0.5 ppm | 0.5 ppm |
| pH | 7.5 | 7.5 |
| Osmolality, mOsm/kg | 240 | 240 |

The contact lens treating solutions of Example 10 and Comparative Example P were tested using the disinfection efficacy test described above. The log reduction values of the 4-hour disinfection efficacy test are shown below in Table 15.

### Testing

TABLE 15

| Ex./Comp. Ex. | S.a. | P.a. | S.m. | C.a. | F.s. |
|---|---|---|---|---|---|
| Ex. 10 | >4.7 | >4.6 | >4.8 | 3.7 | 4.7 |
| Comp. Ex. P | >4.7 | >4.6 | >4.8 | 2.1 | 4.7 |

The contact lens treating solution of Example 10 showed a significantly improved disinfection efficacy for the Candida albicans species as compared to the contact lens treating solution of Comparative Example P.

The following illustrative contact lens treating solutions I-VIII are provided in accordance with non-limiting illustrative embodiments disclosed herein and can be prepared by mixing the following components, listed in Table 16 at amounts per weight. The contact lens treating solutions can have a pH of around 7.7 and an osmolality of around 255 mOsm/kg.

TABLE 16

| Ingredient | I | II | III | IV | V | VI | VII | VIII |
|---|---|---|---|---|---|---|---|---|
| Tris | 0.45% | 0.45% | 0.65% | 0.65% | 0.65% | 0.65% | 0.80% | 0.80% |
| Citric Acid | 0.18% | 0.18% | 0.26% | 0.26% | 0.26% | 0.26% | 0.32% | 0.32% |
| Potassium Citrate | 0.36% | | 0.22% | | | 0.22% | 0.13% | |
| Sodium Citrate | | 0.33% | | 0.20% | 0.20% | | | 0.12% |
| Potassium Chloride | 0.35% | 0.35% | 0.25% | 0.25% | 0.25% | 0.12% | 0.25% | 0.25% |
| Sodium Chloride | | | | | | 0.10% | | |
| Na₂EDTA | 0.10% | 0.10% | 0.10% | 0.10% | 0.10% | 0.10% | 0.10% | 0.10% |
| Glycerol | 0.56% | 0.56% | 0.70% | 0.77% | 0.26% | 0.70% | 0.56% | 0.56% |
| Erythritol | 0.20% | 0.20% | 0.30% | 0.20% | 0.90% | 0.30% | 0.30% | 0.30% |
| Poloxamine 1107 | 1.00% | 1.00% | 0.20% | 1.00% | 0.55% | 1.00% | 1.00% | 0.20% |
| Poloxamer 181 | 0.01% | 0.01% | 0.01% | 0.01% | 0.02% | 0.01% | 0.01% | 0.01% |
| Alexidine, (ppm) | 3.0 | 3.0 | 3.0 | 3.0 | 3.0 | 3.0 | 3.0 | 3.0 |
| PQ-1, (ppm) | 1.5 | 1.5 | 1.5 | 1.5 | 1.5 | 1.5 | 1.5 | 1.5 |

In accordance with an aspect of the invention, a contact lens treating solution, comprises (a) one or more potassium salts selected from the group consisting of potassium chloride, potassium citrate, potassium hydroxide, potassium borate, potassium ethylenediaminetetraacetic acid and potassium phosphate, (b) an antimicrobial agent comprising alexidine or a salt or a free base thereof, and (c) optionally, one or more sodium salts selected from the group consisting of sodium chloride, sodium citrate, sodium hydroxide, sodium phosphate, sodium ethylenediaminetetraacetic acid and sodium borate, wherein the one or more potassium salts are present in an amount greater than an amount of the one or more sodium salts when present.

In one or more additional illustrative embodiments, as may be combined with the preceding paragraphs, the one or more potassium salts include potassium chloride and the corresponding sodium salt is sodium chloride.

In one or more additional illustrative embodiments, as may be combined with the preceding paragraphs, the antimicrobial agent comprises alexidine.

In one or more additional illustrative embodiments, as may be combined with the preceding paragraphs, the contact lens treating solution comprises (a) about 0.02 to about 1.5 wt. % of the one or more potassium salts, based on the total weight of the contact lens treating solution, and (b) about 0.0001 to about 0.0006 wt. % of the antimicrobial agent, based on the total weight of the contact lens treating solution.

In one or more additional illustrative embodiments, as may be combined with the preceding paragraphs, the contact lens treating solution comprises (a) about 0.03 to about 0.9 wt. % of the one or more potassium salts, based on the total weight of the contact lens treating solution, and (b) about 0.0002 to about 0.0003 wt. % of the antimicrobial agent, based on the total weight of the contact lens treating solution.

In one or more additional illustrative embodiments, as may be combined with the preceding paragraphs, the contact lens treating solution further comprises one or more additional antimicrobial agents.

In one or more additional illustrative embodiments, as may be combined with the preceding paragraphs, the one or more additional antimicrobial agents comprise one or more polyquaternium polymers.

In one or more additional illustrative embodiments, as may be combined with the preceding paragraphs, the one or more polyquaternium polymers comprise from about 30 to about 50,000 quaternary-amine-functional repeating units.

In one or more additional illustrative embodiments, as may be combined with the preceding paragraphs, the one or more polyquaternium polymers have a weight average molecular weight Mw of about 3,000 to about 5,000,000.

In one or more additional illustrative embodiments, as may be combined with the preceding paragraphs, the one or more polyquaternium polymers have a weight average molecular weight Mw of about 5,000 to about 40,000.

In one or more additional illustrative embodiments, as may be combined with the preceding paragraphs, the one or more polyquaternium polymers are cationic.

In one or more additional illustrative embodiments, as may be combined with the preceding paragraphs, the one or more polyquaternium polymers comprise Polyquaternium-1.

In one or more additional illustrative embodiments, as may be combined with the preceding paragraphs, the contact lens treating solution comprises about 0.0001 wt. % to about 0.0003 wt. %, based on the total weight of the contact lens treating solution, of the one or more polyquaternium polymers.

In one or more additional illustrative embodiments, as may be combined with the preceding paragraphs, where the one or more additional antimicrobial agents are selected from the group consisting of a polymeric biguanide or a salt or a free base thereof, a terpene compound, a branched, glycerol monoalkyl ether, a branched, glycerol monoalkyl amine, a branched, glycerol monoalkyl sulphide, a fatty acid monoester, wherein the fatty acid monoester comprises an aliphatic fatty acid portion having six to fourteen carbon atoms and an aliphatic hydroxyl portion, an amidoamine compound, and combinations thereof.

In one or more additional illustrative embodiments, as may be combined with the preceding paragraphs, where the polymeric biguanide or a salt or a free base thereof is a polymeric hexamethylene biguanide.

In one or more additional illustrative embodiments, as may be combined with the preceding paragraphs, the contact lens treating solution further comprises one or more surfactants.

In one or more additional illustrative embodiments, as may be combined with the preceding paragraphs, where the one or more surfactants are selected from the group consisting of a poloxamer, a poloxamine and mixtures thereof.

In one or more additional illustrative embodiments, as may be combined with the preceding paragraphs, where the poloxamer is at least one of a poloxamer di(meth)acrylate and a reverse poloxamer di(meth)acrylate, and the poloxamine is at least one of a poloxamine di(meth)acrylate and a reverse poloxamine di(meth)acrylate.

In one or more additional illustrative embodiments, as may be combined with the preceding paragraphs, where the poloxamer is present in the contact lens treating solution in an amount ranging from about 0.001 to about 5.0 wt. %, based on the total weight of the contact lens treating solution, and the poloxamine is present in the contact lens treating solution in an amount ranging from about 0.001 to about 5.0 wt. %, based on the total weight of the contact lens treating solution.

In one or more additional illustrative embodiments, as may be combined with the preceding paragraphs, the contact lens treating solution further comprises one or more comfort agents.

In one or more additional illustrative embodiments, as may be combined with the preceding paragraphs, where the one or more comfort agents are selected from the group consisting of a polyol, an antioxidant and a complex carbohydrate.

In one or more additional illustrative embodiments, as may be combined with the preceding paragraphs, where the polyol is one or more of glycerol and erythritol.

In one or more additional illustrative embodiments, as may be combined with the preceding paragraphs, the contact lens treating solution further comprises one or more polysaccharides.

In one or more additional illustrative embodiments, as may be combined with the preceding paragraphs, where the one or more polysaccharides comprise one or more of an anionic polysaccharide and a non-ionic polysaccharide.

In one or more additional illustrative embodiments, as may be combined with the preceding paragraphs, where the one or more polysaccharides comprise one or more of hyaluronic acid or a salt thereof, chondroitin sulfate, chitosan, aloe vera, carboxymethylcellulose, hemicellulose, hydroxypropyl methyl cellulose, methylcellulose, and ethylcellulose.

In one or more additional illustrative embodiments, as may be combined with the preceding paragraphs, the contact lens treating solution further comprises one or more of a chelating agent, a tonicity adjusting agent, a buffering agent, a pH adjusting agent, a viscosity modifying agent, and a demulcent.

In one or more additional illustrative embodiments, as may be combined with the preceding paragraphs, where the sodium salt includes one or more of sodium chloride, sodium citrate, and sodium phosphate.

In one or more additional illustrative embodiments, as may be combined with the preceding paragraphs, the contact lens treating solution is in the form of an eye care or a contact lens care product selected from the group consisting of eye drops, a contact lens preservative solution, a contact lens cleaning solution, and a contact lens multi-purpose solution.

In one or more additional illustrative embodiments, as may be combined with the preceding paragraphs, the contact lens treating solution is in the form of a multi-purpose solution or rewetting drops.

In accordance with another aspect of the invention, a method of cleaning and disinfecting a contact lens comprises soaking the contact lens in one or more contact lens treating solutions according to the one or more of the illustrative embodiments, as may be combined with the preceding paragraphs, comprising (a) one or more potassium salts selected from the group consisting of potassium chloride, potassium citrate, potassium hydroxide, potassium borate, potassium ethylenediaminetetraacetic acid and potassium phosphate, (b) an antimicrobial agent comprising alexidine or a salt or a free base thereof, and (c) optionally, one or more sodium salts selected from the group consisting of sodium chloride, sodium citrate, sodium hydroxide, sodium phosphate, sodium ethylenediaminetetraacetic acid and sodium borate for a time period sufficient to clean and disinfect the contact lens.

In accordance with yet another aspect of the invention, method for inhibiting adhesion of bacteria to a surface of a contact lens, the method comprising contacting the surface of the contact lens with one or more contact lens treating according to the one or more of the illustrative embodiments, as may be combined with the preceding paragraphs, comprising (a) one or more potassium salts selected from the group consisting of potassium chloride, potassium citrate, potassium hydroxide, potassium borate, potassium ethylenediaminetetraacetic acid and potassium phosphate, (b) an antimicrobial agent comprising alexidine or a salt or a free base thereof, and (c) optionally, one or more sodium salts selected from the group consisting of sodium chloride, sodium citrate, sodium hydroxide, sodium phosphate, sodium ethylenediaminetetraacetic acid and sodium borate.

While compositions and methods are described in terms of “comprising” various components or steps, the compositions and methods can also “consist essentially of” or “consist of” the various components or steps, unless stated otherwise.

The terms “a,” “an,” and “the” are intended to include plural alternatives, e.g., at least one. The terms “including”, “with”, and “having”, as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about”, from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means +20% of the stated value, +15% of the stated value, +10% of the stated value, +5% of the stated value, +3% of the stated value, or +1% of the stated value.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

Various features of the compositions are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

It will be understood that various modifications may be made to the embodiments disclosed herein. Therefore, the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A contact lens treating solution, comprising:
(a) one or more potassium salts selected from the group consisting of potassium chloride, potassium citrate, potassium hydroxide, potassium borate, potassium ethylenediaminetetraacetic acid and potassium phosphate;
(b) an antimicrobial agent comprising alexidine or a salt or a free base thereof; and
(c) optionally, one or more sodium salts selected from the group consisting of sodium sodium citrate, sodium hydroxide, sodium phosphate, sodium chloride, ethylenediaminetetraacetic acid and sodium borate;
wherein the one or more potassium salts are present in an amount greater than an amount of the one or more sodium salts when present.

2. The contact lens treating solution according to claim 1, wherein the one or more potassium salts include potassium chloride and the corresponding sodium salt is sodium chloride.

3. The contact lens treating solution according to claim 1, wherein the antimicrobial agent comprises alexidine.

4. The contact lens treating solution according to claim 1, comprising:
(a) about 0.02 to about 1.5 wt. % of the one or more potassium salts, based on the total weight of the contact lens treating solution; and
(b) about 0.0001 to about 0.0006 wt. % of the antimicrobial agent, based on the total weight of the contact lens treating solution.

5. The contact lens treating solution according to claim 1, further comprising one or more additional antimicrobial agents.

6. The contact lens treating solution according to claim 5, wherein the one or more additional antimicrobial agents comprise one or more polyquaternium polymers.

7. The contact lens treating solution according to claim 6, wherein the one or more polyquaternium polymers comprise from about 30 to about 50,000 quaternary-amine-functional repeating units.

8. The contact lens treating solution according to claim 6, wherein the one or more polyquaternium polymers comprise Polyquaternium-1.

9. The contact lens treating solution according to claim 6, comprising:
about 0.0001 wt. % to about 0.0003 wt. %, based on the total weight of the contact lens treating solution, of the one or more polyquaternium polymers.

10. The contact lens treating solution according to claim 5, wherein the one or more additional antimicrobial agents are selected from the group consisting of a polymeric biguanide or a salt or a free base thereof, a terpene compound, a branched, glycerol monoalkyl ether, a branched, glycerol monoalkyl amine, a branched, glycerol monoalkyl sulphide, a fatty acid monoester, wherein the fatty acid monoester comprises an aliphatic fatty acid portion having six to fourteen carbon atoms and an aliphatic hydroxyl portion, an amidoamine compound, and combinations thereof.

11. The contact lens treating solution according to claim 10, wherein the polymeric biguanide or a salt or a free base thereof is a polymeric hexamethylene biguanide.

12. The contact lens treating solution according to claim 1, further comprising one or more surfactants.

13. The contact lens treating solution according to claim 12, wherein the one or more surfactants are selected from the group consisting of a poloxamer, a poloxamine and mixtures thereof.

14. The contact lens treating solution according to claim 1, further comprising one or more comfort agents.

15. The contact lens treating solution according to claim 14, wherein the one or more comfort agents are selected from the group consisting of a polyol, an antioxidant and a complex carbohydrate.

16. The contact lens treating solution according to claim 1, further comprising one or more polysaccharides.

17. The contact lens treating solution according to claim 1, wherein the sodium salt includes one or more of sodium chloride, sodium citrate, and sodium phosphate.

18. The contact lens treating solution according to claim 1, in the form of an eye care or a contact lens care product selected from the group consisting of eye drops, a contact lens preservative solution, a contact lens cleaning solution, and a contact lens multi-purpose solution.

19. A method of cleaning and disinfecting a contact lens, the method comprising soaking the contact lens in one or more contact lens treating solutions for a time period sufficient to clean and disinfect the contact lens, wherein the one or more contact lens treating solutions comprise:
(a) one or more potassium salts selected from the group consisting of potassium chloride, potassium citrate, potassium hydroxide, potassium borate, potassium ethylenediaminetetraacetic acid and potassium phosphate;
(b) an antimicrobial agent comprising alexidine or a salt or a free base thereof; and
(c) optionally, one or more sodium salts selected from the group consisting of sodium sodium citrate, sodium hydroxide, sodium phosphate, sodium chloride, ethylenediaminetetraacetic acid and sodium borate;
wherein the one or more potassium salts are present in an amount greater than an amount of the one or more sodium salts when present.

20. A method for inhibiting adhesion of bacteria to a surface of a contact lens, the method comprising contacting the surface of the contact lens with one or more contact lens treating solutions comprising:
(a) one or more potassium salts selected from the group consisting of potassium chloride, potassium citrate, potassium hydroxide, potassium borate, potassium ethylenediaminetetraacetic acid and potassium phosphate;
(b) an antimicrobial agent comprising alexidine or a salt or a free base thereof; and
(c) optionally, one or more sodium salts selected from the group consisting of sodium chloride, sodium citrate, sodium hydroxide, sodium phosphate, sodium ethylenediaminetetraacetic acid and sodium borate;
wherein the one or more potassium salts are present in an amount greater than an amount of the one or more sodium salts when present.
